(19) United States
(12) Patent Application Publication
(10) Pub. No.: US 2016/0002684 A1
(43) Pub. Date: Jan. 7, 2016

(54) Method for Producing L-Lysine by Modifying Aconitase Gene and/or Regulatory Elements thereof

(71) Applicant: NINGXIA EPPEN BIOTECH CO., LTD, Yinchuan (CN)

(72) Inventors: Jiyin MA, Yinchuan (CN); Tingyi WEN, Yinchuan (CN); Jinlong CHEN, Yinchuan (CN); Yong LIANG, Yinchuan (CN); Shuwen LIU, Yinchuan (CN); Aiying WEI, Yinchuan (CN); Lipeng YANG, Yinchuan (CN); Rui REN, Yinchuan (CN); Gang MENG, Yinchuan (CN); Chunguang ZHAO, Yinchuan (CN); Yun ZHANG, Yinchuan (CN); Xiuling SHANG, Yinchuan (CN); Xiaowei GUO, Yinchuan (CN)

(21) Appl. No.: 14/384,370

(22) PCT Filed: Jan. 7, 2014
(86) PCT No.: PCT/CN2014/070228

§ 371 Date: Sep. 11, 2014

(30) Foreign Application Priority Data
Feb. 8, 2013 (CN) 201310050144.3
Feb. 8, 2013 (CN) 201310050196.0

(51) Int. Cl. C12P 13/08; C12N 9/88

(52) U.S. Cl. CPC C12P 13/08; C12N 9/88; C12Y 402/01003

(57) ABSTRACT

A method for producing L-lysine by fermentation comprises the steps of modifying an aconitase gene and/or regulatory element thereof in a chromosome of a bacterium so that the activity and/or the expression amount of the aconitase of the bacterium are reduced but not eliminated; and producing L-lysine by the fermentation of the modified bacterium. In addition, also provided are methods and uses derived from the method as well as bacteria used in these methods and uses.

This application is the U.S. national phase of International Application No. PCT/CN2014/070228 Filed 7 Jan. 2014 which designated the U.S. and claims priority to Chinese Application Nos. CN201310050196.0, CN201310050144.3 filed on 8 Feb. 2013, the entire contents of each of which are hereby incorporated by reference.

### TECHNICAL FIELD

The present invention belongs to the field of amino acid fermentation, and specifically relates to a method of producing L-lysine by fermentation and methods and uses derived from the method as well as bacteria used in these methods and uses.

### BACKGROUND ART

To produce L-lysine by fermentation with L-lysine-producing bacteria (e.g., E. coli of Escherichia and bacteria of Corynebacterium) has been already widely industrially used. These bacteria may be isolated from the natural environment, and/or may be obtained by mutagenesis or genetically engineered modification. Prior arts focus on the genetically engineered modifications of genes such as pnt, dap, ppc and the like, and pay no attention to a regulatory element of a gene encoding an aconitase (e.g., aconitase A from Escherichia coli, an aconitase from Corynebacterium glutamicum or Corynebacterium pekinense) for the production of L-lysine.

An aconitase is an enzyme of the tricarboxylic acid cycle, which catalyzes two-step chemical reactions: the transformation of citric acid to aconitic acid and the transformation of aconitic acid of isocitric acid. In the known bacteria of Escherichia, the gene acnA, the nucleotide sequence of which is shown in SEQ ID No: 1, encodes aconitase A. However, perhaps due to its metabolite far away from the final product L-lysine as well as many and complex intermediary metabolism branches, its role in the L-lysine fermentation has not been suggested. And there are no reports about the effects of an aconitase from other L-lysine-producing bacteria such as Corynebacterium glutamicum and the like on the L-lysine fermentation. The aconitases described in documents such as Chinese patents CN1289368A and CN101631871A relate to the fermentation of L-glutamic acid, and are not taught to have any effects on the fermentation of L-lysine.

After a long research and practice, especially by virtue of some luck, the inventors fortuitously found that a modification of an aconitase gene and regulatory element thereof is conducive to the increase of an L-lysine yield.

In addition, in prior arts, an expression amount and/or enzyme activity are increased by the increase of copy number and the introduction of site-directed mutation of a useful enzyme gene, or an enzyme activity and/or expression amount are eliminated by the knockout of a harmful enzyme gene. However the inventors found that an aconitase gene and regulatory element thereof are different from those in prior arts and can not be simply increased or knocked out. Especially the elimination of the expression of the gene acnA by the knockout results in slow growth of bacteria and difficulty for a practical use, so a novel method of modifying an aconitase gene and/or regulatory element thereof for increasing an L-lysine yield was invented.

Furthermore, the method does not interfere in modification sites in chromosomes of a great amount of L-lysine-producing bacteria modified according to prior arts, so as to further increase the effect and be useful for practically producing L-lysine by using a great variety of bacteria.

### DISCLOSURE OF THE INVENTION

The problem to be solved by the invention is to provide a novel method of producing L-lysine by fermentation and related methods including a method of increasing a fermentation yield of L-lysine compared to an unmodified bacterium, a use of a modified bacterium for producing L-lysine by fermentation, a use of a modified bacterium for increasing a fermentation yield of L-lysine compared to an unmodified bacterium, and/or a method of modifying a bacterium. Furthermore, the invention also provides a polynucleotide, vector and/or bacterium used in the methods mentioned above.

Specifically, in first aspect, the invention provides a method of producing L-lysine by fermentation, which comprises the steps of:

(1) modifying an aconitase gene and/or regulatory element thereof in a chromosome of a bacterium so that the activity and/or the expression amount of the aconitase of the bacterium are reduced but not eliminated; and
(2) producing L-lysine by fermentation with the bacterium obtained by the modification of step (1).

The term “modification” used herein means a change of an object in need of modification resulting in a certain effect. Modification techniques of a gene or a regulatory element in a chromosome include, but are not limited to, mutagenesis, site-directed mutation and/or homologous recombination, preferably the last two. To modify a gene or a regulatory element in a chromosome is an addition, deletion or substitution of one or more nucleotides in the nucleotide sequence of the gene or the regulatory element. These techniques are widely described in documents of molecular biology and microbiology, and many of them have been already commercially available. In the embodiments of the invention, the modification can be carried out according to the principle of homologous recombination by using the pKOV plasmid commercially available by Addgene or using the pK18mobsacB plasmid, so that an unmodified aconitase gene and/or regulatory element thereof in a bacterial chromosome are modified to be a novel aconitase gene and/or regulatory element thereof, and the expression amount and/or the enzyme activity of the aconitase in the modified bacterium are reduced but not eliminated. Therefore preferably the modification used herein is a modification by homologous recombination.

After a long research, the inventors found that in Escherichia coli, bacteria of Corynebacterium and the like, the elimination of the expression of an aconitase, by using the technique such as the knockout of a gene, results in slow growth of bacteria and even no amino acids produced. Therefore the modification of the invention does reduce and does not eliminate the expression amount of the aconitase of the bacterium obtained by the modification compared to the bacterium without the modification. Preferably the expression amount of aconitase A of the bacterium obtained by the modification is reduced by 20%-95%, more preferably by 50%-90%, e.g. by 65%, 70% or 80%.

Accordingly, the invention also provides other uses or methods. For example, in second aspect, the invention provides a method of increasing a fermentation yield of L-lysine, which comprises the steps of:

(1) modifying an aconitase gene and/or regulatory element thereof in a chromosome of a bacterium so that the activity and/or the expression amount of the aconitase of the bacterium are reduced but not eliminated; and
(2) producing L-lysine by fermentation with the bacterium obtained by the modification of step (1).

L-lysine is an important metabolite of a bacterium, and most of bacteria can generate more or less amount of L-lysine. Although an L-lysine-producing bacterium with low yield is not suitable for cost-efficiently producing L-lysine, yet the methods of the invention can result in the increase of L-lysine and can be used in a cost-insensitive area. Undoubtedly a preferable bacterium used herein is an L-lysine-producing bacterium with high yield, and its yield can be further increased by using the methods of the invention. In addition, in the methods or uses of the invention, except a modification of an aconitase gene and/or regulatory element thereof in a chromosome of a bacterium, no other modifications may be carried out. For example, an aconitase gene in a bacterial chromosome may not be modified while a regulatory element of the aconitase gene is modified, and vice versa. For example, only one of an aconitase gene and regulatory element thereof in a chromosome of a bacterium, especially an L-lysine-producing bacterium with high yield, may be modified.

For example, in third aspect, the invention provides a use of a bacterium obtained by a modification for producing L-lysine by fermentation, wherein the modification is to modify an aconitase gene and/or regulatory element thereof in a chromosome of a bacterium, and the enzyme activity and/or the expression amount of the aconitase of the bacterium obtained by the modification are reduced but not eliminated.

The bacterium obtained by the modification may be used alone, with other L-lysine-producing bacteria, or by other means for producing L-lysine by fermentation. Unless otherwise defined (e.g. without the definition of “obtained by the modification”), the term “bacterium” used herein means an unmodified bacterium or a bacterium without the modification, an aconitase gene and a regulatory element located around the locus of the gene in a chromosome of which are an aconitase gene and a regulatory element without the decrease of the enzyme activity and/or the expression amount of the aconitase, e.g., an aconitase gene and a regulatory element from a wild-type bacterium.

For example, in fourth aspect, the invention provides a use of a bacterium obtained by a modification for increasing a fermentation yield of L-lysine, wherein the modification is to modify an aconitase gene and/or regulatory element thereof in a chromosome of a bacterium, and the enzyme activity and/or the expression amount of the aconitase of the bacterium obtained by the modification are reduced but not eliminated.

As used herein, a bacterium may be an L-lysine producing bacterium, e.g. a bacterium of Escherichia or Corynebacterium. In one preferable aspect, a bacterium may be a bacterium of Escherichia, more preferably Escherichia coli, e.g. filial strains of Escherichia coli K-12 including strains derived from the strain W3110; In the other preferable aspect, a bacterium may be a bacterium of Corynebacterium, more preferably Corynebacterium glutamicum or Corynebacterium pekinense. Because few prior arts suggest the role of an aconitase gene and/or regulatory element thereof from a bacterium in the production/fermentation of L-lysine and focus on the loci such as pnt, dap, ppc and the like to modify the genes in chromosomes, there are no reports about a modification of an aconitase gene and/or regulatory element therearound from L-lysine-producing bacteria (especially bacteria of Escherichia or Corynebacterium, e.g. E. coli, Corynebacterium glutamicum or Corynebacterium pekinense) in prior arts, and thus the L-lysine-producing bacteria substantially have wild-type aconitase genes and regulatory elements therearound. So they may be modified by using the methods of the invention for the increase of a fermentation yield of L-lysine. In the embodiments of the invention, all of L-lysine-producing bacteria with low or high yield can be modified by using the methods of the invention to increase a fermentation yield of L-lysine.

Substantially, in fifth aspect, the invention provides a method of modifying a bacterium, which comprises the step of modifying an aconitase gene and/or regulatory element thereof in a chromosome of a bacterium so that the activity and/or the expression amount of the aconitase of the bacterium obtained by the modification are reduced but not eliminated.

The bacterium obtained by the method of the fifth aspect of the invention can be used for producing or generating L-lysine by fermentation. Therefore in sixth aspect, the invention provides a bacterium obtained by the method of the fifth aspect of the invention.

According to the experiments by the inventors, many of nucleotide sequences of aconitase genes (acnA) from bacteria of Escherichia (e.g., E. coli) are shown in SEQ ID No: 1, while many of nucleotide sequences of aconitase genes from bacteria of Corynebacterium (e.g., Corynebacterium glutamicum or Corynebacterium pekinense) are shown in SEQ ID No: 2. To modify these aconitase genes so as to reduce but not eliminate the enzyme activity and/or the expression of the aconitases in the modified bacteria, can increase a fermentation yield of L-lysine. Therefore in the embodiments of the invention, the nucleotide sequence of the aconitase gene is shown in SEQ ID No: 1 or 2. For other bacteria, the nucleotide sequences of aconitase genes from the bacteria can be obtained by the techniques such as sequencing and comparing identities of sequences for the modification of the methods of the invention.

In the invention, the preferable step of modifying an aconitase gene in a chromosome of a bacterium is an addition, deletion or substitution of one or more nucleotides in the nucleotide sequence of the aconitase gene, as long as the enzyme activity and/or the expression amount of the aconitase from the modified bacterium are reduced but not eliminated.

According to the experience of the inventors, although it is usually difficult to increase an activity and/or an expression amount of a bacterium's enzyme since the bacterium has been evolved for a long time and modification sites for the increase need be researched particularly, yet the research for the decrease of the activity and/or the expression amount of the enzyme is much easier. For example, the nucleotide sequence out of domains of an enzyme may be mutated. In the invention, the preferable step of modifying an aconitase gene in a chromosome of a bacterium may be a substitution of one or more nucleotides in the nucleotide sequence of the aconitase gene. For example, the substitution includes a substitution for the initiation codon of the aconitase gene, preferably a substitution of GTG. It can be carried out for Escherichia coli, Corynebacterium glutamicum, Corynebacterium pekinense and the like. Also in the invention, the preferable step of modifying an aconitase gene in a chromosome of a bacterium may be a deletion of one or more nucleotides in the nucleotide sequence of the aconitase gene. For example, the deletion includes a deletion in the nucleotide sequence of the aconitase gene, preferably a deletion of 1-120 nucleotides, more preferably a deletion of 1-90 nucleotides, most preferably a deletion of 90 nucleotides, e.g. a deletion of 90 nucleotides before the termination codon in the nucleotide sequence of the aconitase gene. It can be carried out for Escherichia coli, Corynebacterium glutamicum, Corynebacterium pekinense and the like.

As used herein, a regulatory element means a polynucleotide located upstream or downstream of a gene (e.g., an aconitase gene) and regulating the transcription and/or the expression of the gene in order to have an effect on the expression amount of the gene. It may include non-coding and encoding sequences. A regulatory element may be a promoter, enhancer, repressor or other polynucleotide related to transcription and/or expression control. A preferable regulatory element may be a promoter. In one embodiment of the invention, the nucleotide sequence of the promoter is shown in SEQ ID No: 4 or 6. A preferable regulatory element may also be a repressor, for example a transcription repressor. In one embodiment of the invention, the nucleotide sequence of the transcription repressor is shown in SEQ ID No: 7. By modifying a promoter and/or repressor, an enzyme activity and/or expression amount of an aconitase in a modified bacterium are reduced but not eliminated.

In the invention, the preferable step of modifying a regulatory element of an aconitase gene in a chromosome of a bacterium is an addition, deletion or substitution of one or more nucleotides in the nucleotide sequence of the regulatory element of the aconitase gene, as long as the enzyme activity and/or the expression amount of the aconitase from the modified bacterium are reduced but not eliminated.

According to the experience of the inventors, although it is usually difficult to increase a transcription activity of a bacterium's promoter or enhancer since the bacterium has been evolved for a long time and modification sites for the increase need be researched particularly, yet the research for the decrease of the transcription activity of the promoter or enhancer is much easier. For example, one or several nucleotide sequence can be added for increasing the distance between a promoter and the initiation codon of a gene, or a promoter with weak transcription activity is substituted for an original promoter. In the invention, the preferable step of modifying a regulatory element of an aconitase gene in a chromosome of a bacterium may be a substitution of one or more nucleotides in a promoter of the aconitase gene, for example a substitution of a promoter with weak transcription activity, more preferably a substitution of the nucleotide sequence shown in SEQ ID No: 3 or 5.

Furthermore, in the invention, the preferable step of modifying a regulatory element of an aconitase gene in a chromosome of a bacterium may be an addition of the nucleotide sequence of a transcription repressor for the aconitase gene, for example an addition of the nucleotide sequence of a new transcription repressor or increase of the copy number of the nucleotide sequence of an original transcription repressor. A transcription repressor may be added behind a promoter (especially a promoter with strong transcription activity) in order to increase its expression amount and inhibit the transcription of an aconitase gene more efficiently. In an embodiment of the invention, the nucleotide sequence shown in SEQ ID No: 8 and 7 in tandem is added.

Furthermore, the invention also provides intermediate products such as a polynucleotide and/or vector used in the methods mentioned above, as well as uses of the products. For example, in seventh aspect, the invention provides a polynucleotide, the nucleotide sequence of which is selected from

(a) the nucleotide sequence obtained by a substitution (preferably of GTG) for the initiation codon of the nucleotide sequence shown in SEQ ID No: 1;
(b) the nucleotide sequence obtained by a deletion (preferably of 1-120 nucleotides, more preferably of 1-90 nucleotides, most preferably of 90 nucleotides) in the nucleotide sequence shown in SEQ ID No: 1 or 2, e.g. a deletion of 90 nucleotides before the termination codon in the nucleotide sequence shown in SEQ ID No: 1 or 2; and
(c) the nucleotide sequence shown in SEQ ID No: 8 or 7 in tandem.

In eighth aspect, the invention provides a vector, which comprises the polynucleotide of the seventh aspect of the invention.

In ninth aspect, the invention provides a use of the polynucleotide of the seventh aspect of the invention and/or the vector of the eighth aspect of the invention in the method or use of the first, second, third and/or fourth aspect of the invention. That is that in the methods or uses of the first, second, third and/or fourth aspects of the invention, the polynucleotide of the seventh aspect of the invention and/or the vector of the eighth aspect of the invention are used.

In tenth aspect, the invention provides a use of the polynucleotide of the seventh aspect of the invention and/or the vector of the eighth aspect of the invention in the preparation of the bacterium of the fifth aspect of the invention. That is that in the preparation process of the bacterium of the fifth aspect of the invention, the polynucleotide of the seventh aspect of the invention and/or the vector of the eighth aspect of the invention are used.

The beneficial effects of the invention include that novel techniques are invented and proved for increasing a fermentation yield of L-lysine when using L-lysine-producing bacteria with high and low yield; and do not interfere in modification sites in chromosomes of a great amount of L-lysine-producing bacteria with high yield modified according to prior arts, so as to further increase the yield, be useful for practically producing L-lysine by the fermentation of bacteria, and be easy to popularize.

For a better understanding of the invention, it will now be described in greater detail by reference to specific examples. It should be noted that the examples only exemplify the invention, and should not be construed as limiting the scope of the invention. According to the description of the invention, various modifications and alterations of the invention are obvious to a person skilled in the art.

In addition, the publications cited in the invention are used to illustrate the invention, the contents of which are incorporated herein by reference, as if they have been written down herein.

### EXAMPLES

Embodiments of the invention are further exemplified by the following examples. Unless otherwise specified, the techniques used in the examples are well-known for a person skilled in the art and commercially available devices and reagents (see Molecular Cloning: A Laboratory Manual (3^{rd} Edition), Science Press, Microbiology Experiments (4^{th} Edition), Higher Education Press, and the manufacturer's instructions of the devices and reagents).

### Example 1

### Substitution of GTG for the Initiation Codon ATG of Aconitase

The genomic chromosome of the wild-type Escherichia coli strain, E. coli K12 W3110 (commercially available from Biological Resource Center, National Institute of Technology and Evaluation (NITE Biological Resource Center, NBRC)), was extracted as a template for a PCR amplification, by using the primers P1/P2 and P3/P4 respectively. Two DNA fragments of 510 bp and 620 bp were obtained and named Up1 and Down1 fragments respectively. The process of the PCR amplification was shown as follows: denaturalizing for 30 s (seconds) in 94° C., annealing for 30 s (seconds) in 52° C., extending for 30 s (seconds) in 72° C., and performing for 30 times circularly. And the sequences of the primers were shown as follows:

| | |
|---|---|
| P1: | 5′-CGCGGATCCGGAGTCGTCACCATTATGCC-3′ |
| P2: | 5′-TCTCGTAGGGTTGACGACACAGCTCCTCCTTAATGACAGG-3′ |
| P3: | 5′-CCTGTCATTAAGGAGGAGCTGTGTCGTCAACCCTACGAGA-3′ |
| P4: | 5′-ATTGCGGCCGCTCCATTCACCGTCCTGCAAT-3′ |

The two DNA fragments were purified by agarose gel electrophoresis and mixed as a template for overlap PCR amplification by using the primers P1/P4. An approximately 1200 bp fragment was obtained and named Up-Down1 fragment. The process of the PCR amplification was shown as follows: denaturalizing for 30 s (seconds) in 94° C., annealing for 30 s (seconds) in 52° C., extending for 60 s (seconds) in 72° C., and performing for 30 times circularly.

The Up-Down1 purified by agarose gel electrophoresis and the pKOV plasmid (commercially available from Addgene) were digested respectively by Bam HI/Not I. The digestion products were purified by agarose gel electrophoresis and ligated to be a vector pKOV-Up-Down1 for further transform. The vector pKOV-Up-Down1 was determined by sequencing the vector by a sequencing company to have a correct point mutation (A-G) in the gene fragment of acnA, and stored for further use.

The constructed plasmid pKOV-Up-Down1 was transformed by electroporation into the L-lysine-producing strain with low yield, E. coli NRRL B-12185 (commercially available from Agricultural Research Service Culture Collection (NRRL); see also U.S. Pat. No. 4,346,170A for its construction method), and the L-lysine-producing strain with high yield, E. coli K12 W3110 Δ3 (commercially available from Institute of Microbiology, Chinese Academy of Sciences; the strain is an L-lysine-producing engineering strain mutagenized and mutated from E. coli K12 W3110) respectively. The two strains were determined by sequencing to have the wild-type gene of acnA (i.e., sites 1333855 to 1336530 of GenBank accession number U00096.2) and upstream and downstream elements thereof in their chromosomes. According to the manufacturer's instruction of pKOV plasmid from Addgene, homologous recombination-positive clones were selected after a recovery culture in LB medium under the conditions of 30° C. and 100 rpm for 2 h, and were determined by sequencing to have the mutation of the initiation codon ATG to GTG in the wild-type gene of acnA in their chromosomes. Finally both of L-lysine-producing E. coli strains with low and high yield having the mutation of the initiation codon of acnA were obtained and named YP-13633 and YP-13664 respectively. The expression amount of aconitases in the two modified strains was measured and reduced by approximately 75˜85% (different values in different media).

### Example 2

### Mutation of the Aconitase Gene Sequence for the Decrease of the Activity of Aconitase

(1) Construction of E. coli Strains

90 nucleotides before the termination codon in the gene acnA of E. coli were deleted for the decrease of the aconitase activity. Specifically, the genomic chromosome of the wild-type Escherichia coli strain, E. coli K12 W3110, was extracted as a template for PCR amplification, by using the primers P5/P6 and P7/P8 respectively. Two DNA fragments of 752 bp and 657 bp were obtained and named Up2 and Down2 fragments respectively. The process of the PCR amplification was shown as follows: denaturalizing for 30 s (seconds) in 94° C., annealing for 30 s (seconds) in 52° C., extending for 30 s (seconds) in 72° C., and performing for 30 times circularly. And the sequences of the primers were shown as follows:

| | |
|---|---|
| P5: | 5′-CGCGGATCCCGTCACACGATCCGATACCT-3′ |
| P6: | 5′-CGGCAAGCAAATAGTTGTTATACGACTTCCTGGCTACCAT-3′ |
| P7: | 5′-ATGGTAGCCAGGAAGTCGTATAACAACTATTTGCTTGCCG-3′ |
| P8: | 5′-ATTGCGGCCGC CATGGGGCGATTTCCTGATG-3′ |

The two DNA fragments were purified by agarose gel electrophoresis and mixed as a template for an overlap PCR amplification by using the primers P5/P8. An approximately 1400 bp fragment was obtained and named Up-Down2 fragment. The process of the PCR amplification was shown as follows: denaturalizing for 30 s (seconds) in 94° C., annealing for 30 s (seconds) in 52° C., extending for 60 s (seconds) in 72° C., and performing for 30 times circularly.

The Up-Down2 purified by agarose gel electrophoresis and the pKOV plasmid (commercially available from Addgene) were digested respectively by Bam HI/Not I. The digestion products were purified by agarose gel electrophoresis and ligated to be a vector pKOV-Up-Down2 for further transform. The vector pKOV-Up-Down2 was determined by sequencing the vector by a sequencing company to have a deletion of 90 bp bases before the termination codon in the gene fragment of acnA, and stored for further use.

According to the manufacturer's instruction of pKOV plasmid from Addgene, the constructed plasmid pKOV-Up-Down2 was transformed by electroporation into the L-lysine-producing strain with low yield, E. coli NRRL B-12185 (see also U.S. Pat. No. 4,346,170A for its construction method), and the L-lysine-producing strain with high yield, E. coli K12 W3110 Δ3 respectively. The two strains were determined by sequencing to have the wild-type gene of acnA and upstream and downstream elements thereof in their chromosomes. Homologous recombination-positive clones were selected, and were determined by sequencing to have the deletion of 90 bp bases before the termination codon in the gene of acnA in their chromosomes. Finally both of L-lysine-producing E. coli strains with low and high yield, in which the enzyme activities of acnA were reduced, were obtained and named YP-13675 and YP-13699 respectively. The aconitase activities in the two modified strains were measured and reduced by approximately 60˜80% (different values in different media).

### (2) Construction of Corynebacterium Strains

90 nucleotides before the termination codon in the acn gene of Corynebacterium were deleted for the decrease of the aconitase activity. The process of construction was substantially the same as that of step (1) mentioned above, except that the genome of Corynebacterium pekinense AS1.299 (which is similar to Corynebacterium glutamicum and is sometimes mistakenly regarded as Corynebacterium glutamicum; commercially available from China General Microbiological Culture Collection Center (CGMCC)) was used as a template for an amplification by using the primers P9˜P12 as follows (corresponding to the primers P4˜P8 mentioned above respectively), and a 542 bp fragment (Up2), a 527 bp fragment (Down2), and an approximately 1069 bp fragment (Up-Down) were obtained. And the sequences of the primers were shown as follows:

| | |
|---|---|
| P9: | 5′-CGGGATCCTGCAGCTCAGTACTTGGAT-3′ |
| P10: | 5′-AAAGTCTTCTAATTAC TTACTGCGTCGAACTCGACG-3′ |
| P11: | 5′-GTTCGACGCAGTAAGTAATTAGAAGACTTTTGAT-3′ |
| P12: | 5′-TCCCCCGGGGAATACCGGGTCGGTGCG-3′ |

Then the Up-Down2 fragment purified by agarose gel electrophoresis and pK18mobsacB plasmid (commercially available from American Type Culture Collection (ATCC)) were digested respectively by Bam HI/Sma I. The digestion products were purified by agarose gel electrophoresis and ligated to be a recombinant vector pK18mobsacB-Up-P-Down having a deletion of 90 bp bases before the termination codon in the acn gene. The constructed plasmid pK18mobsacB-Up-Down2 was verified by sequencing and transformed respectively by electroporation into the L-lysine-producing strain with low yield, Corynebacterium pekinense AS1.299, the L-lysine-producing strain with low yield, Corynebacterium glutamicum ATCC 13032 (commercially available from ATCC), and the L-lysine-producing strain with high yield, Corynebacterium pekinense AS1.563 (commercially available from China General Microbiological Culture Collection Center). The last two strains were determined by sequencing to have in their chromosomes the acn gene from Corynebacterium pekinense AS1.299, the nucleotide sequence of which is shown in SEQ ID NO: 2. Homologous recombination-positive clones were selected after a recovery culture in BHIS medium under the conditions of 30° C. and 120 rpm for 2 h, and were verified by sequencing. Finally three L-lysine-producing Corynebacterium strains with low, low and high yield having the mutation of the acn gene were obtained and named YP-14808, YP-14852 and YP-14837.

### Example 3

### Substitution of a Promoter with Weak Transcription Activity for the Promoter of Aconitase Gene

(1) Construction of E. coli Strains

By the analysis of the upstream sequence of acnA in E. coli K12 W3110, we provided a promoter with weak transcription activity (the sequence of which is shown in SEQ ID No: 3) to replace the region of the wild-type promoter (the sequence of which is shown in SEQ ID No: 4) in the upstream of the ORF of the acnA gene for the decrease of the expression of the wild-type acnA gene.

Specifically, the genomic chromosome of the wild-type Escherichia coli strain, E. coli K12 W3110, was extracted as a template for PCR amplification, by using the primers P13/P14 and P15/P16 respectively. Two DNA fragments of 580 bp and 618 bp were obtained and named Up3 and Down3 fragments respectively. A plasmid comprising the promoter with weak transcription activity mentioned above was used as a template for PCR amplification by using P17/P18, and a 161 bp promoter fragment with weak transcription activity was obtained and named P fragment. The process of the PCR amplification was shown as follows: denaturalizing for 30 s (seconds) in 94° C., annealing for 30 s (seconds) in 52° C., extending for 30 s (seconds) in 72° C., and performing for 30 times circularly.

The three DNA fragments mentioned above were purified by agarose gel electrophoresis. Then the Up3 and P fragments were mixed as a template for overlap PCR amplification by using the primers P13/P18. An approximately 716 bp fragment was obtained and named Up-P fragment. The process of the PCR amplification was shown as follows: denaturalizing for 30 s (seconds) in 94° C., annealing for 30 s (seconds) in 52° C., extending for 60 s (seconds) in 72° C., and performing for 30 times circularly.

The Up-P and Down3 fragments purified by agarose gel electrophoresis were mixed as a template for an overlap PCR amplification by using the primers P13/P16. An approximately 1334 bp fragment was obtained and named Up-P-down fragment. The process of the PCR amplification was shown as follows: denaturalizing for 30 s (seconds) in 94° C., annealing for 30 s (seconds) in 52° C., extending for 60 s (seconds)) in 72° C., and performing for 30 times circularly.

The sequences of the primers mentioned above were shown as follows:

| | |
|---|---|
| P13: | 5′-CGCGGATCCGAAGAAATTGAGGTCATGTT-3′ |
| P14: | 5′-GGTTTCTTAGACGTCGGATTCGTTTCGTTTCTGTTTCAT |
| | T-3′ |
| P15: | 5′-ATCAGCAGGACGCACTGACCCATTAAGGAGGAGCTATGT |
| | CG-3′ |
| P16: | 5′-ATTGCGGCCGCTCCATTCACCGTCCTGCAATT-3′ |
| P17: | 5′-AATGAAACAGAAACGAAACGCAATCCGACGTCTAAGAAA |
| | CC-3′ |
| P18: | 5′-CGACATAGCTCCTCCTTAATGGGTCAGTGCGTCCTGCTG |
| | AT-3′ |

The Up-P-down purified by agarose gel electrophoresis and the pKOV plasmid (commercially available from Addgene) were digested respectively by Bam HI/Not I. The digestion products were purified by agarose gel electrophoresis and ligated to be a vector pKOV-Up-P-Down for further transform. The vector pKOV-Up-P-Down was determined by sequencing the vector by a sequencing company to have a correct sequence of the promoter with weak transcription activity, and stored for further use.

The constructed plasmid pKOV-Up-P-Down was transformed by electroporation into the L-lysine-producing strain with low yield, E. coli NRRL B-12185, and the L-lysine-producing strain with high yield, E. coli K12 W3110 Δ3 respectively. The two strains were determined by sequencing to have the wild-type gene of acnA and upstream and downstream elements thereof in their chromosomes, where the upstream promoter comprises sites form 2102518 to 2102713 of GenBank accession number CP004009.1. According to the manufacturer's instruction of pKOV plasmid from Addgene, homologous recombination-positive clones were selected after a recovery culture in LB medium under the conditions of 30° C. and 100 rpm for 2 h, and were determined by sequencing to have the substitution of the promoter with weak transcription activity for the wild-type promoter in the upstream of the gene acnA in their chromosomes.

Finally both of L-lysine-producing E. coli strains with low and high yield having the mutation of the promoter of acnA were obtained and named YP-13627 and YP-13682 respectively. The expression amount of aconitases in the two modified strains was measured and reduced by 65˜80% in different media.

### (2) Construction of Corynebacterium Strains

By the analysis of the upstream sequence of acn in Corynebacterium, we provided a promoter with weak transcription activity (the sequence of which is shown in SEQ ID No: 5) to replace the region of the 166 bp wild-type promoter (the sequence of which is shown in SEQ ID No: 6) in the upstream of the ORF of the acn gene from Corynebacterium. The process of construction was substantially the same as that of step (1) mentioned above, except that:

The genome of Corynebacterium pekinense AS1.299 (commercially available from China General Microbiological Culture Collection Center) was used as a template for an amplification by using the primers P19˜P24 as follows (corresponding to the primers P13˜P18 mentioned above respectively), and a 573 bp fragment (Up3), a 581 bp fragment (Down3), a 130 bp fragment (P), and an approximately 1284 bp fragment (Up-P-down) were obtained. And the sequences of the primers were shown as follows;

| | |
|---|---|
| P19: | 5′-CGGGATCCGCCAAAGCAACCAACCCC-3′ |
| P20: | 5′-CTTTTTAGTTTTCAACGGTCGGATTTGCTCGAAAT-3′ |
| P21: | 5′-GCCGAAAC AAAGTAGCCGAAGCAGACGCCGTCG-3′ |
| P22: | 5′-CGGAATTCTGACCTGGTGGACGATAC-3′ |
| P23: | 5′-CGAGCAAATCCGACCGTTGAAAACTAAAAAGCTGG-3′ |
| P24: | 5′-GCGTCTGCTTCGGCTACTTTGTTTCGGCCACCC-3′ |

Then the Up-P-down fragment purified by agarose gel electrophoresis and the pK18mobsacB plasmid were digested respectively by Bam HI/Eco RI. The digestion products were purified by agarose gel electrophoresis and ligated to be a recombinant vector pK18mobsacB-Up-P-Down for further substitution of the promoter. The constructed plasmidpK18mobsacB-Up-P-Down was verified by sequencing and transformed respectively by electroporation into the L-lysine-producing strain with low yield, Corynebacterium pekinense AS1.299, the L-lysine-producing strain with low yield, Corynebacterium glutamicum ATCC 13032, and the L-lysine-producing strain with high yield, Corynebacterium pekinense AS1.563. The last two strains were determined by sequencing to have in their chromosomes the acn gene and upstream and downstream elements thereof from Corynebacterium pekinense AS1.299.

Homologous recombination-positive clones were selected after a recovery culture in BHIS medium under the conditions of 30° C. and 120 rpm for 2 h, and were verified by sequencing. Finally three L-lysine-producing Corynebacterium strains with low, low and high yield having the mutation of the promoter of acn were obtained and named YP-14755, YP-14732 and YP-14780.

### Example 4

### Increase of the Copy Number of the Gene acnR Encoding the Transcription Repressor of Aconitase Gene

The copy number of the gene acnR encoding the transcription repressor of acn was increased for the decrease of the transcription level of the acn gene. Specifically, the genome of Corynebacterium pekinense AS1.299 was used as a template for PCR amplification by using the primers P25/P26 and P27/P28 respectively. Two DNA fragments of 715 bp and 797 bp were obtained and named Up4 and Down4 fragments. The template was used or a PCR amplification by using the primers P29/P30. A 567 bp acnR fragment was obtained and named R fragment, the nucleotide sequence of which is shown in SEQ ID No: 7. In addition, the expression plasmid pXMJ19 (commercially available from Biovector Science Lab, Inc) was used as a template for a PCR amplification by using the primers P31 [symbol] P32. An 164 bp promoter P_{tac} with strong transcription activity was obtained and named P_{tac} fragment, the nucleotide sequence of which is shown in SEQ ID No: 8. The process of the PCR amplification was shown as follows: denaturalizing for 30 s (seconds) in 94° C., annealing for 30 s (seconds) in 52° C., extending for 30 s (seconds) in 72° C., and performing for 30 times circularly.

The sequences of the primers mentioned above were shown as follows:

| | |
|---|---|
| P25: | 5′-CGGGATCCTTCGCAACCGATAGAGCA-3′ |
| P26: | 5′-CACGAATTATGCAGAATAAGCCTTTAAGTAACAA-3′ |
| P27: | 5′-TAAACGCGACTAAGCGTGACCATTAAAAGGCT-3′ |
| P28: | 5′-CGGAATTCAAAAGCCTATTAAGTGTC-3′ |
| P29: | 5′-TTCACACAGGAAAGTGTCCGTAGCGGCAGGCGA-3′ |
| P30: | 5′-TTTAATGGTCACGC TTAGTCGCGTTTACGGACAG-3′ |
| P31: | 5′-TTAAAGGCTTATTCTGCATAATTCGTGTCGCTC-3′ |
| P32: | 5′-GCCGCTACGGACACTTTCCTGTGTGAAATTGTTA-3′ |

The R and P_{tac} fragments were purified by agarose gel electrophoresis and mixed as a template for an overlap PCR amplification by using the primers P31/P30. An approximately 73 lbp fragment was obtained and named P_{tac}-R fragment. The Up4 and P_{tac}-R fragments were purified by agarose gel electrophoresis and mixed as a template for overlap PCR amplification by using the primers P25/P30. An approximately 1446 bp fragment was obtained and named Up4-P_{tac}-R fragment. The Up4-P_{tac}-R and Down4 fragments were purified by agarose gel electrophoresis and mixed as a template for an overlap PCR amplification by using the primers P25/P28. An approximately 2243 bp fragment was obtained and named Up-P_{tac}-R-Down fragment. The process of the PCR amplification was shown as follows: denaturalizing for 30 s (seconds) in 94° C., annealing for 30 s (seconds) in 52° C., extending for 60 s (seconds) in 72° C., and performing for 30 times circularly.

Then the Up-P_{tac}-R-Down purified by agarose gel electrophoresis and the pK18mobsacB plasmid were digested respectively by Bam HI/Eco RI. The digestion products were purified by agarose gel electrophoresis and ligated to be a recombinant vector pK18mobsacB-Up-P_{tac}-R-Down for further insertion of additional copies of acnR into a non-coding region of a chromosome. The vector pK18mobsacB-Up-P_{tac}-R-Down was determined by sequencing the vector by a sequencing company to have the P_{tac}-acnR gene fragments, and stored for further use.

The constructed plasmid pK18mobsacB-Up-P_{tac}-R-Down was transformed respectively by electroporation into the L-lysine-producing strain with low yield, Corynebacterium pekinense AS1.299, the L-lysine-producing strain with low yield, Corynebacterium glutamicum ATCC 13032, and the L-lysine-producing strain with high yield, Corynebacterium pekinense AS1.563. Homologous recombination-positive clones were selected after a recovery culture in BHIS medium under the conditions of 30° C. and 100 rpm for 2 h, and were verified by sequencing. Finally three L-lysine-producing Corynebacterium strains with low, low and high yield, into non-coding regions of the chromosomes of which additional copies of the gene acnR were inserted, were obtained and named YP-14857, YP-14896 and YP-14860.

### Example 5

### L-Lysine Fermentation Experiments

For the fermentation of E. coli, 25 mL of the seed medium shown in table 1 was inoculated with each of the strains E. coli K12 W3110 Δ3, E. coli NRRL B-12185 and E. coli strains constructed by examples 1˜3 respectively, and cultured under the conditions of 37° C. and 220 rpm for 9 h. Then 25 mL of the fermentation medium shown in table 1 was inoculated with lmL culture product of the seed medium, and cultured under the conditions of 37° C. and 220 rpm for 48 h. The yield of L-lysine was measured by HPLC after the culture process.

TABLE 1
Culture medium for E. coli

| | Seed | Fermentation |
|---|---|---|
| | medium | medium |
| (Ingredient) | (g/L) | (g/L) |
| glucose | 15 | 40 |
| ammonium sulphate | 4 | 10 |
| potassium dihydrogen phosphate | 3 | 1.6 |
| magnesium sulphate heptahydrate | 0.4 | 1 |
| ferrous sulphate heptahydrate | 0.01 | 0.03 |
| manganese sulfate monohydrate | 0.01 | 0.03 |
| yeast extract | 2.0 | 4.0 |
| calcium carbonate | | 25 |
| KOH | pH 7.0 | pH 7.0 |
| L-tyrosine | | 0.1 |
| L-methionine | | 0.5 |
| L-threonine | | 0.1 |
| L-isoleucine | | 0.05 |

For the fermentation of Corynebacterium, 30 mL of the seed medium shown in table 2 was inoculated with each of the strains Corynebacterium AS1.299, ATCC13032, AS1.563 and Corynebacterium strains constructed by examples 2˜4 respectively, and cultured under the conditions of 30° C. and 220 rpm for 8 h. Then 30 mL of the fermentation medium shown in table 2 was inoculated with 1 mL culture product of the seed medium, and cultured under the conditions of 30° C. and 220 rpm for 48 h. The yield of L-lysine was measured by HPLC after the culture process.

TABLE 2
Culture medium for Corynebacterium

| | Seed | Fermentation |
|---|---|---|
| | medium | medium |
| (Ingredient) | (g/L) | (g/L) |
| glucose | 20 | 40 |
| ammonium sulphate | 5 | 20 |
| potassium dihydrogen phosphate | 1 | 1.6 |
| magnesium sulphate heptahydrate | 0.7 | 0.8 |
| ferrous sulphate heptahydrate | 0.01 | 0.01 |
| manganese sulfate monohydrate | 0.01 | 0.01 |
| yeast extract | 5.0 | 4.0 |
| calcium carbonate | | 20 |
| KOH | pH 7.0 | pH 7.0 |

The results are show in table 3. To mutate (e.g., delete or replace) the aconitase structure of the strains of Escherichia coli and Corynebacterium for the decrease of the activity of the enzyme, or to modify (e.g., replace and insert) the regulatory elements of the aconitase genes of the strains of Escherichia coli and Corynebacterium for the decrease of the expression amount of the enzyme, is conducive to the increase of L-lysine yield.

TABLE 3
Yield of L-Lysine by Strains

| | Yield of | Yield increase |
|---|---|---|
| Strain | L-lysine (g/L) | ratio (%) |
| E. coli NRRL B-12185 | 1.5 | — |
| YP-13633 | 2.1 | 40 |
| YP-13675 | 1.8 | 20 |
| YP-13627 | 2.0 | 33 |
| E. coli K12 W3110 Δ3 | 10.2 | — |
| YP-13664 | 16.1 | 57.8 |
| YP-13699 | 12.5 | 22.5 |
| YP-13682 | 14.1 | 38.2 |
| Corynebacterium AS1.299 | 1.2 | — |
| YP-14808 | 1.6 | 33 |
| YP-14755 | 1.9 | 58 |
| YP-14857 | 1.4 | 17 |
| Corynebacterium ATCC 13032 | 1.1 | — |
| YP-14852 | 1.5 | 36 |
| YP-14732 | 2.0 | 82 |
| YP-14896 | 1.3 | 18 |
| Corynebacterium AS1.563 | 23.5 | — |
| YP-14837 | 27.4 | 16.6 |
| YP-14780 | 31.2 | 32.8 |
| YP-14860 | 25.6 | 8.9 |

1. A method of producing L-lysine by fermentation or of increasing a fermentation yield of L-lysine, which comprises the steps of:
(1) modifying an aconitase gene and/or regulatory element thereof in a chromosome of a bacterium so that the activity and/or the expression amount of the aconitase of the bacterium are reduced but not eliminated; and
(2) producing L-lysine by fermentation with the bacterium obtained by the modification of step (1).

2. A use of a bacterium obtained by a modification for producing L-lysine by fermentation or for increasing a fermentation yield of L-lysine, wherein the modification is to modify an aconitase gene and/or regulatory element thereof in a chromosome of a bacterium, and the activity and/or the expression amount of the aconitase of the bacterium obtained by the modification are reduced but not eliminated.

3. The method according to claim 1, which comprises the step of modifying an aconitase gene and/or regulatory element thereof in a chromosome of a bacterium so that the activity and/or the expression amount of the aconitase of the bacterium obtained by the modification are reduced but not eliminated.

4. The method according to 3, wherein the step of modifying an aconitase gene in a chromosome of a bacterium is an addition, deletion or substitution of one or more nucleotides in the nucleotide sequence of the aconitase gene.

5. The method according to claim 4, wherein the substitution includes a substitution for the initiation codon of the aconitase gene, preferably a substitution of GTG.

6. The method according to claim 4, wherein the deletion includes a deletion in the nucleotide sequence of the aconitase gene, preferably a deletion of 1-120 nucleotides, more preferably a deletion of 1-90 nucleotides, most preferably a deletion of 90 nucleotides, e.g. a deletion of 90 nucleotides before the termination codon in the nucleotide sequence of the aconitase gene.

7. The method according to claim 6, wherein the nucleotide sequence of the aconitase gene is shown in SEQ ID No: 1 or 2.

8. The method according to claim 1, wherein the step of modifying a regulatory element of an aconitase gene in a chromosome of a bacterium is an addition, deletion or substitution of one or more nucleotides in the nucleotide sequence of the regulatory element of the aconitase gene.

9. The method according to claim 8, wherein the regulatory element is a promoter, preferably the nucleotide sequence of the promoter is shown in SEQ ID No: 4 or 6.

10. The method according to claim 8, wherein the substitution includes a substitution for the nucleotide sequence of a promoter of the aconitase gene, preferably a substitution of the nucleotide sequence shown in SEQ ID No: 3 or 5.

11. The method according to claim 8, wherein the regulatory element is a transcription repressor, preferably the nucleotide sequence of the transcription repressor is shown in SEQ ID No: 7.

12. The method according to claim 8, wherein the addition includes an addition in the nucleotide sequence of a transcription repressor of the aconitase gene, preferably an addition of the nucleotide sequence shown in SEQ ID No: 8 and 7 in tandem.

13. The method according to claim 12, wherein the bacterium is an Escherichia or Corynebacterium bacterium, preferably Escherichia coli, Corynebacterium glutamicum or Corynebacterium pekinense.

14. A bacterium obtained by the method according to claim 13.

15. A polynucleotide, the nucleotide sequence of which is selected from
(a) the nucleotide sequence obtained by a substitution (preferably of GTG) for the initiation codon of the nucleotide sequence shown in SEQ ID No: 1;
(b) the nucleotide sequence obtained by a deletion (preferably of 1-120 nucleotides, more preferably of 1-90 nucleotides, most preferably of 90 nucleotides) in the nucleotide sequence shown in SEQ ID No: 1 or 2, e.g. a deletion of 90 nucleotides before the termination codon in the nucleotide sequence shown in SEQ ID No: 1 or 2; and
(c) the nucleotide sequence shown in SEQ ID No: 8 or 7 in tandem.

16. A vector, which comprises the polynucleotide of claim 15.

17. The use according to claim 2, use of the polynucleotide of claim 15 and/or the vector of claim 16.

18. The use according to claim 2, the polynucleotide of claim 15 and/or the vector of claim 16 in the preparation of the bacterium according to claim 14.


SEQUENCE LISTING (39 sequences)

SEQ ID NO: 1 - DNA, 2676 bp, Escherichia coli
atgtcgtcaaccctacgagaagccagtaaggacacgttgcaggccaaagataaaacttaccactactacagcctgccgcttgctgctaaatcactgggcgatatcacccgtctacccaagtcactcaaagttttgctcgaaaacctgctgcgctggcaggatggtaactcggttaccgaagaggatatccacgcgctggcaggatggctgaaaaatgcccatgctgaccgtgaaattgcctaccgcccggcaagggtgctgatgcaggactttaccggcgtacctgccgttgttgatctggcggcaatgcgcgaagcggttaaacgcctcggcggcgatactgcaaaggttaacccgctctcaccggtcgacctggtcattgaccactcggtgaccgtcgatcgttttggtgatgatgaggcatttgaagaaaacgtacgcctggaaatggagcgcaaccacgaacgttatgtgttcctgaaatggggaaagcaagcgttcagtcggtttagcgtcgtgccgccaggcacaggcatttgccatcaggttaacctcgaatatctcggcaaagcagtgtggagtgaattgcaggacggtgaatggattgcttatccggatacactcgttggtactgactcgcacaccaccatgatcaacggccttggcgtgctggggtggggcgttggtgggatcgaagcagaagccgcaatgttaggccagccggtttccatgcttatcccggatgtagtgggcttcaaacttaccggaaaattacgtgaaggtattaccgccacagacctggttctcactgttacccaaatgctgcgcaaacatggcgtggtggggaaattcgtcgaattttatggtgatggtctggattcactaccgttggcggatcgcgccaccattgccaatatgtcgccagaatatggtgccacctgtggcttcttcccaatcgatgctgtaaccctcgattacatgcgtttaagcgggcgcagcgaagatcaggtcgagttggtcgaaaaatatgccaaagcgcagggcatgtggcgtaacccgggcgatgaaccaatttttaccagtacgttagaactggatatgaatgacgttgaagcgagcctggcagggcctaaacgcccacaggatcgcgttgcactgcccgatgtaccaaaagcatttgccgccagtaacgaactggaagtgaatgccacgcataaagatcgccagccggtcgattatgttatgaacggacatcagtatcagttacctgatggcgctgtggtcattgctgcgataacctcgtgcaccaacacctctaacccaagtgtgctgatggccgcaggcttgctggcgaaaaaagccgtaactctgggcctcaagcggcaaccatgggtcaaagcgtcgctggcaccgggttcgaaagtcgtttctgattatctggcaaaagcgaaactgacaccgtatctcgacgaactggggtttaaccttgtgggatacggttgtaccacctgtattggtaactctgggccgctgcccgatcctatcgaaacggcaatcaaaaaaagcgatttaaccgtcggtgcggtgctgtccggcaaccgtaactttgaaggccgtatccatccgctggttaaaactaactggctggcctcgccgccgctggtggttgcctatgcgctggcgggaaatatgaatatcaacctggcttctgagcctatcggccatgatcgcaaaggcgatccggtttatctgaaagatatctggccatcggcacaagaaattgcccgtgcggtagaacaagtctccacagaaatgttccgcaaagagtacgcagaagtttttgaaggcacagcagagtggaagggaattaacgtcacacgatccgatacctacggttggcaggaggactcaacctatattcgcttatcgcctttctttgatgaaatgcaggcaacaccagcaccagtggaagatattcacggtgcgcggatcctcgcaatgctgggggattcagtcaccactgaccatatctctccggcgggcagtattaagcccgacagcccagcgggtcgatatctacaaggtcggggtgttgagcgaaaagactttaactcctacggttcgcggcgtggtaaccatgaagtgatgatgcgcggcaccttcgccaatattcgcatccgtaatgaaatggtgcctggcgttgaaggggggatgacgcggcatttacctgacagcgacgtagtctctatttatgatgctgcgatgcgctataagcaggagcaaacgccgctggcggtgattgccgggaaagagtatggatcaggctccagtcgtgactgggcggcaaaaggtccgcgtctgcttggtattcgtgtggtgattgccgaatcgtttgaacgaattcaccgttcgaatttaattggcatgggcatcctgccgctggaatttccgcaaggcgtaacgcgtaaaacgttagggctaaccggggaagagaagattgatattggcgatctgcaaaacctacaacccggcgcgacggttccggtgacgcttacgcgcgcggatggtagccaggaagtcgtaccctgccgttgtcgtatcgacaccgcgacggagttgacctactaccagaacgacggcattttgcattatgtcattcgtaatatgttgaagtaa

SEQ ID NO: 2 - DNA, 2832 bp, Corynebacterium glutamicum
ttggagctcactgtgactgaaagcaagaactccttcaatgctaagagcacccttgaagttggcgacaagtcctatgactacttcgccctctctgcagtgcctggcatggagaagctgccgtactccctcaaggttctcggagagaaccttcttcgtaccgaagacggcgcaaacatcaccaacgagcacattgaggctatcgccaactgggatgcatcttccgatccaagcatcgaaatccagttcaccccagcccgtgttctcatgcaggacttcaccggtgtcccttgtgtagttgacctcgcaaccatgcgtgaggcagttgctgcactcggtggcgaccctaacgacgtcaacccactgaacccagccgagatggtcattgaccactccgtcatcgtggaggctttcggccgcccagatgcactggctaagaacgttgagatcgagtacgagcgcaacgaggagcgttaccagttcctgcgttggggttccgagtccttctccaacttccgcgttgttcctccaggaaccggtatcgtccaccaggtcaacattgagtacttggctcgcgtcgtcttcgacaacgagggccttgcatacccagatacctgcatcggtaccgactcccacaccaccatggaaaacggcctgggcatcctgggctggggcgttggtggcattgaggctgaagcagcaatgctcggccagccagtgtccatgctgatccctcgcgttgttggcttcaagttgaccggcgagatcccagtaggcgttaccgcaactgacgttgtgctgaccatcaccgaaatgctgcgcgaccacggcgtcgtccagaagttcgttgagttctacggctccggtgttaaggctgttccactggctaaccgtgcaaccatcggcaacatgtccccagagttcggctccacctgtgcgatgttcccaatcgacgaggagaccaccaagtacctgcgcctcaccggccgcccagaagagcaggttgcactggtcgaggcttacgccaaggcgcagggcatgtggctcgacgaggacaccgttgaagctgagtactccgagtacctcgagctggacctgtccaccgttgttccttccatcgctggccctaagcgcccacaggaccgcatccttctctccgaggcaaaggagcagttccgtaaggatctgccaacctacaccgacgacgctgtttccgtagacacctccatccctgcaacccgcatggttaacgaaggtggcggacagcctgaaggcggcgtcgaagctgacaactacaacgcttcctgggctggctccggcgagtccttggctactggcgcagaaggacgtccttccaagccagtcaccgttgcatccccacagggtggcgagtacaccatcgaccacggcatggttgcaattgcatccatcacctcttgcaccaacacctctaacccatccgtgatgatcggcgctggcctgatcgcacgtaaggcagcagaaaagggcctcaagtccaagccttgggttaagaccatctgtgcaccaggttcccaggttgtcgacggctactaccagcgcgcagacctctggaaggaccttgaggccatgggcttctacctctccggcttcggctgcaccacctgtattggtaactccggcccactgccagaggaaatctccgctgcgatcaacgagcacgacctgaccgcaaccgcagttttgtccggtaaccgtaacttcgagggacgtatctcccctgacgttaagatgaactacctggcatccccaatcatggtcattgcttacgcaatcgctggcaccatggacttcgacttcgagaacgaagctcttggacaggaccaggacggcaacgacgtcttcctgaaggacatctggccttccaccgaggaaatcgaagacaccatccagcaggcaatctcccgtgagctttacgaagctgactacgcagatgtcttcaagggtgacaagcagtggcaggaactcgatgttcctaccggtgacaccttcgagtgggacgagaactccacctacatccgcaaggcaccttacttcgacggcatgcctgtcgagccagtggcagtcaccgacatccagggcgcacgcgttctggctaagctcggcgactctgtcaccaccgaccacatctcccctgcttcctccattaagccaggtacccctgcagctcagtacttggatgagcacggtgtggaacgccacgactacaactccctgggttccaggcgtggtaaccacgaggtcatgatgcgcggcaccttcgccaacatccgcctccagaaccagctggttgacatcgcaggtggctacacccgcgacttcacccaggagggtgctccacaggcgttcatctacgacgcttccgtcaactacaaggctgctggcattccgctggtcgtcttgggcggcaaggagtacggcaccggttcttcccgtgactgggcagctaagggcactaacctgctcggaattcgcgcagttatcaccgagtccttcgagcgtattcaccgctccaacctcatcggtatgggcgttgtcccactgcagttccctgcaggcgaatcccacgagtccctgggccttgacggcaccgagaccttcgacatcaccggactgaccgcactcaacgagggcgagactcctaagactgtcaaggtcaccgcaaccaaggagaacggcgacgtcgtcgagttcgacgcagttgtccgcatcgacaccccaggtgaggctgactactaccgccacggcggcatcctgcagtacgtgctgcgtcagatggctgcttcttctaagtaa

SEQ ID NO: 3 - DNA, 161 bp, Escherichia coli
caatccgacgtctaagaaaccattattatcatgacattaacctataaaaataggcgtatcacgaggccctttcgtcttcacctcgagtccctatcagtgatagagatggacatccctatcagtgatagagatactgagcacatcagcaggacgcactgacc

SEQ ID NO: 4 - DNA, 454 bp, Escherichia coli
ggaatgttccgtcgttattccagacgactggcaactaacatcgcagcagcaagcctttatagaactgtttgctgaagatgatcagccgaaacaataattatcatcattcttattacccatttttaatgaattaaagggcttttaatacaccgcagcaataacagcttgagttatctcaacacaaaataataaccgttaagggtgtagcctatgatcaacacaaatatgaaatattggtcctggatgggcgcgttttctctgtcgatgctcttctgggccgaactcctctggatcattactcactgatccttgaccccgctgcggcggggttgtcatttgctttgccacaaggtttctcctcttttatcaatttgggttgttatcaaatcgttacgcgatgtttgtgttatctttaatattcaccctgaagagaatcagggcttcgcaaccctgt

SEQ ID NO: 5 - DNA, 130 bp, Corynebacterium glutamicum
ccgttgaaaactaaaaagctgggaaggtgaatcgaatttcggggctttaaagcaaaaatgaacagcttggtctatagtggctaggtaccctttttgttttggacacatgtagggtggccgaaacaaagta

SEQ ID NO: 6 - DNA, 166 bp, Corynebacterium glutamicum
acgcgccaagaaccccaactttcccgccagaacgcttgtactgttaggataatgaagacgtagggtccttttccacagttctgtggaatgagaatccgatgtttttctcacgccggctcagccgaagcagacgccgtcgcgaaatctcaccctaaaaaagttagaa

SEQ ID NO: 7 - DNA, 567 bp, Corynebacterium glutamicum
gtgtccgtagcggcaggcgacaaaccaacaaatagccgtcaagaaatcctcgaaggtgcccgacggtgcttcgctgagcacggctatgaaggcgcaaccgtacgccgactggaagaagcaacaggtaaatcacgcggagcgatctttcatcacttcggtgacaaagaaaacctgttcctagccctcgcgcgggaagatgcagcccgcatggcggaggtggtgtctgaaaatggcctcgttgaagtgatgcgaggaatgctggaagatcctgaacgatatgactggatgtcagtacgcctggagatctccaagcagctgcgcaccgacccggtattccgcgcaaaatggattgatcaccaaagtgttctagacgaagctgtccgcgtgcgtttgtcccgcaacgtggataagggacaaatgcgcactgacgtcccgatcgaagtgctgcacaccttcttagagactgttctcgacggtttcatctcccgtcttgctaccggcgcatccacagaaggactgtccgaagtattggatctggtcgagggaactgtccgtaaacgcgactaa

SEQ ID NO: 8 - DNA, 164 bp, Corynebacterium glutamicum
ctgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaa

SEQ ID NO: 9 - DNA, 29 bp, Artificial Sequence
  Primer for PCR
cgcggatccggagtcgtcaccattatgcc

SEQ ID NO: 10 - DNA, 40 bp, Artificial Sequence
  Primer for PCR
tctcgtagggttgacgacacagctcctccttaatgacagg

SEQ ID NO: 11 - DNA, 40 bp, Artificial Sequence
  Primer for PCR
cctgtcattaaggaggagctgtgtcgtcaaccctacgaga

SEQ ID NO: 12 - DNA, 31 bp, Artificial Sequence
  Primer for PCR
attgcggccgctccattcaccgtcctgcaat

SEQ ID NO: 13 - DNA, 29 bp, Artificial Sequence
  Primer for PCR
cgcggatcccgtcacacgatccgatacct

SEQ ID NO: 14 - DNA, 40 bp, Artificial Sequence
  Primer for PCR
cggcaagcaaatagttgttatacgacttcctggctaccat

SEQ ID NO: 15 - DNA, 40 bp, Artificial Sequence
  Primer for PCR
atggtagccaggaagtcgtataacaactatttgcttgccg

SEQ ID NO: 16 - DNA, 31 bp, Artificial Sequence
  Primer for PCR
attgcggccgccatggggcgatttcctgatg

SEQ ID NO: 17 - DNA, 27 bp, Artificial Sequence
  Primer for PCR
cgggatcctgcagctcagtacttggat

SEQ ID NO: 18 - DNA, 36 bp, Artificial Sequence
  Primer for PCR
aaagtcttctaattacttactgcgtcgaactcgacg

SEQ ID NO: 19 - DNA, 34 bp, Artificial Sequence
  Primer for PCR
gttcgacgcagtaagtaattagaagacttttgat

SEQ ID NO: 20 - DNA, 27 bp, Artificial Sequence
  Primer for PCR
tcccccggggaataccgggtcggtgcg

SEQ ID NO: 21 - DNA, 29 bp, Artificial Sequence
  Primer for PCR
cgcggatccgaagaaattgaggtcatgtt

SEQ ID NO: 22 - DNA, 40 bp, Artificial Sequence
  Primer for PCR
ggtttcttagacgtcggattcgtttcgtttctgtttcatt

SEQ ID NO: 23 - DNA, 41 bp, Artificial Sequence
  Primer for PCR
atcagcaggacgcactgacccattaaggaggagctatgtcg

SEQ ID NO: 24 - DNA, 32 bp, Artificial Sequence
  Primer for PCR
attgcggccgctccattcaccgtcctgcaatt

SEQ ID NO: 25 - DNA, 41 bp, Artificial Sequence
  Primer for PCR
aatgaaacagaaacgaaacgcaatccgacgtctaagaaacc

SEQ ID NO: 26 - DNA, 41 bp, Artificial Sequence
  Primer for PCR
cgacatagctcctccttaatgggtcagtgcgtcctgctgat

SEQ ID NO: 27 - DNA, 26 bp, Artificial Sequence
  Primer for PCR
cgggatccgccaaagcaaccaacccc

SEQ ID NO: 28 - DNA, 33 bp, Artificial Sequence
  Primer for PCR
gccgaaacaaagtagccgaagcagacgccgtcg

SEQ ID NO: 29 - DNA, 26 bp, Artificial Sequence
  Primer for PCR
cggaattctgacctggtggacgatac

SEQ ID NO: 30 - DNA, 35 bp, Artificial Sequence
  Primer for PCR
cgagcaaatccgaccgttgaaaactaaaaagctgg

SEQ ID NO: 31 - DNA, 33 bp, Artificial Sequence
  Primer for PCR
gcgtctgcttcggctactttgtttcggccaccc

SEQ ID NO: 32 - DNA, 25 bp, Artificial Sequence
  Primer for PCR
gggatccttcgcaaccgatagagca

SEQ ID NO: 33 - DNA, 34 bp, Artificial Sequence
  Primer for PCR
cacgaattatgcagaataagcctttaagtaacaa

SEQ ID NO: 34 - DNA, 32 bp, Artificial Sequence
  Primer for PCR
taaacgcgactaagcgtgaccattaaaaggct

SEQ ID NO: 35 - DNA, 26 bp, Artificial Sequence
  Primer for PCR
cggaattcaaaagcctattaagtgtc

SEQ ID NO: 36 - DNA, 33 bp, Artificial Sequence
  Primer for PCR
ttcacacaggaaagtgtccgtagcggcaggcga

SEQ ID NO: 37 - DNA, 34 bp, Artificial Sequence
  Primer for PCR
tttaatggtcacgcttagtcgcgtttacggacag

SEQ ID NO: 38 - DNA, 33 bp, Artificial Sequence
  Primer for PCR
ttaaaggcttattctgcataattcgtgtcgctc

SEQ ID NO: 39 - DNA, 34 bp, Artificial Sequence
  Primer for PCR
gccgctacggacactttcctgtgtgaaattgtta